(19) United States
(12) Patent Application Publication
(10) Pub. No.: US 2014/0316003 A1
(43) Pub. Date: Oct. 23, 2014

(54) METHODS AND COMPOSITIONS FOR TREATING OR PREVENTING ATTENTION DEFICIT HYPERACTIVITY DISORDER

(71) Applicant: Omthera Pharmaceuticals, Inc., Bedminster, NJ (US)

(72) Inventors: Michael H. Davidson, Highland Park, IL (US); Gerald L. Wisler, Windemere, FL (US)

(73) Assignee: Omthera Pharmaceuticals, Inc., Bedminster, NJ (US)

(21) Appl. No.: 14/352,972

(22) PCT Filed: Oct. 19, 2012
(86) PCT No.: PCT/US2012/061131

§ 371 Date: Apr. 18, 2014

Related U.S. Application Data
(60) Provisional application No. 61/549,917, filed on Oct. 21, 2011.

(51) Int. Cl. C12Q 1/68; A61K 9/00; A23L 1/30; A61K 31/202

(52) U.S. Cl. CPC C12Q 1/6883; A61K 31/202; A61K 9/006; A23L 1/3008
USPC 514/560; 506/9; 435/6.12; 436/94

(57) ABSTRACT

The present disclosure relates to methods of identifying subjects who are suffering from, or are susceptible to developing, ADHD. The methods comprise determining whether the subject is an efficient converter of medium chain polyunsaturated fatty acids to long-chain polyunsaturated fatty acids. Also provided are methods of treating ADHD in a subject, comprising administering to the subject an effective amount of a composition comprising long chain omega-3 fatty acids.

### 1. CROSS-REFERENCE TO RELATED APPLICATIONS

This application claims the benefit under 35 U.S.C. §119(e) of U.S. Provisional Application No. 61/549,917, filed Oct. 21, 2011, herein incorporated by reference in its entirety.

### 2. BACKGROUND

Attention deficit hyperactivity disorder (ADHD) is one of the most common developmental disorders in children. Symptoms include age-inappropriate difficulty staying focused and paying attention, difficulty controlling behavior and hyperactivity. Evidence suggests that symptoms may persist into adolescence and adulthood for up to 50% of children diagnosed with ADHD and may lead to poor outcomes, including social and emotional adjustment problems, poor acceptance by peers, personality trait disorders, antisocial behavior and substance abuse. Although the exact causes of ADHD are unknown, it has been postulated that the disorder results from a combination of factors, including environment, nutrition, brain injury and genetics. See Attention Deficit Hyperactivity Disorder (ADHD), National Institutes of Health Publication No. 08-3572 (2008).

Essential fatty acids are known to play a role in cognition, learning and behavior in animals. Researchers have observed that animals on diets restricted in omega-3 fatty acids have lower brain composition of omega-3 fatty acids and evidence cognitive and behavioral deficits. See, e.g., Yamamoto et al. (1988) J. Lipid Res. 29:1013-1021; Wainwright et al. (1994) Dev. Psychobiol. 27(7):467-487. Children diagnosed with ADHD have been found to have significantly lower amounts of omega-3 fatty acids in red blood cells than control subjects even though no dietary deficit of omega-3 fatty acids was observed in the ADHD subjects. See, e.g., Chen et al. (2004) J. Nutritional Biochem. 15:467-472. More recently, researchers have found a significant association of ADHD with a single nucleotide polymorphism in a fatty acid desaturase gene that encodes an enzyme involved in omega-3 and omega-6 fatty acid synthesis. See Brookes et al. (2006) Biol. Psychiatry 60:1053-1061.

Diagnosis of ADHD is clinical and is based on comprehensive medical, developmental, educational, and psychological evaluations using, for example, the DSM-IV-TR Symptom Criteria. Because symptoms of ADHD can occur from time to time in everyone, can vary from person to person or can be present in other developmental disorders, ADHD is difficult to diagnose. Many experts think that ADHD is overdiagnosed because the criteria are applied inaccurately.

Although certain symptoms and signs of ADHD tend to diminish with age, there is no cure. The disorder is managed by treatments aimed at relieving the symptoms so that patients can be successful in school and lead productive lives. Current treatments include medication, behavior modification, life-style changes and counseling.

There is, therefore, a need for additional methods by which to identify patients who have, or who are susceptible to, ADHD. There is also a need for additional methods of treating ADHD.

### 3. SUMMARY

The inventors have discovered that certain subjects suffering from, or susceptible to developing, ADHD are efficient converters of medium chain polyunsaturated fatty acids (“mc-PUFAs”) to long chain polyunsaturated fatty acids (“lc-PUFAs”). An efficient converter, as described in more detail below, is a subject who more efficiently produces long chain polyunsaturated fatty acid products from dietary medium chain fatty acids than a subject who is not an efficient converter.

Accordingly, provided herein are methods of identifying subjects who are suffering from, or who are susceptible to developing ADHD, comprising determining whether the subject is an efficient converter of mc-PUFAs to lc-PUFAs. Efficient converter status may be determined phenotypically, genotypically, or by combining phenotypic and genotypic determinations.

The inventors have further discovered that ADHD can be treated or prevented in such efficient converters by administering an effective amount of compositions comprising omega-3 lc-PUFAs.

Thus, methods are provided herein for treating or preventing ADHD in a subject who is an efficient converter of mc-PUFAs to lc-PUFAs. The methods comprise administering to a subject who has been determined to be an efficient converter of mc-PUFAs to lc-PUFAs an amount of a composition comprising omega-3 lc-PUFAs effective to treat ADHD. Methods are also provided for treating ADHD in a subject in need thereof, comprising: (a) determining whether the subject is an efficient converter of mc-PUFA to lc-PUFA; and, for subjects determined to be an efficient converter of mc-PUFA to lc-PUFA (b) administering to the subject an effective amount of an ADHD therapy and adjunctively administering an amount of a composition comprising omega-3 lc-PUFAs effective to treat ADHD to the subject.

In particular embodiments, the composition comprises omega-3 PUFAs in free acid form. In certain embodiments, eicosapentaenoic acid (EPA) and docosahexaenoic acid (DHA) are in the free acid form, and EPA is present in an amount of from about 50% to about 60% by weight and DHA is present in an amount of from about 15% to about 25% by weight.

In certain embodiments, the efficient converter is on an ADHD therapy. In other embodiments, an ADHD therapy is clinically indicated for the subject.

In various embodiments, the methods further comprise a step of monitoring the ic-PUFA levels in the blood of the subject. In particular embodiments, methods described herein further comprise a step of monitoring the lc-PUFA levels in the blood of the subject and a step of adjusting the dosage of omega-3 long chain polyunsaturated fatty acids based on the lc-PUFA levels in the blood of the subject.

It should be noted that the indefinite articles “a” and “an” and the definite article “the” are used in the present application to mean one or more unless the context clearly dictates otherwise. Further, the term “or” is used in the present application to mean the disjunctive “or” or the conjunctive “and.”

All publications mentioned in this specification are herein incorporated by reference. Any discussion of documents, acts, materials, devices, articles or the like that has been included in this specification is solely for the purpose of providing a context for the present disclosure. It is not to be taken as an admission that any or all of these matters form part of the prior art or were common general knowledge in the field relevant to the present disclosure as it existed anywhere before the priority date of this application.

The features and advantages of the disclosure will become further apparent from the following detailed description of embodiments thereof.

### 4. BRIEF DESCRIPTION OF THE FIGURES

FIG. 1 shows the known metabolic pathway of conversion of the dietary fatty acids linoleic acid (an omega-6 fatty acid) and α-linolenic acid (an omega-3 fatty acid) to long chain polyunsaturated fatty acids (“lc-PUFAs”) in the human body.

### 5. DETAILED DESCRIPTION

The inventors have discovered that certain subjects suffering from, or susceptible to, ADHD are efficient converters of medium chain polyunsaturated fatty acids (“mc-PUFAs”) to long chain polyunsaturated fatty acids (“lc-PUFAs”).

Accordingly, provided herein are methods of identifying subjects who suffer from, or are susceptible to, ADHD, comprising determining whether the subject is an efficient converter of mc-PUFAs to lc-PUFAs. Efficient converter status may be determined phenotypically, genotypically, or by combining phenotypic and genotypic determinations.

5.1. Determining “Efficient Converter” Status

The term “polyunsaturated fatty acid” as used herein refers to a compound having the formula:

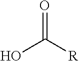

wherein R represents a C18 to C24 carbon chain with two or more double bonds. A mc-PUFA is a fatty acid that has a carbon chain (R) with up to 18 carbons. A lc-PUFA is a fatty acid that has a carbon chain (R) with 20 or more carbons. Polyunsaturated fatty acids can be denominated as “Ca:b”, wherein “a” is an integer that represents the total number of carbon atoms and “b” is an integer that refers to the number of double bonds in the carbon chain.

Two series of polyunsaturated fatty acids are relevant herein: omega-3 polyunsaturated fatty acids and omega-6 polyunsaturated fatty acids. The term “omega-3 fatty acid” as used herein refers to a polyunsaturated fatty acid wherein the first double bond is located after the third carbon in the carbon chain (R), numbering from the free methyl end of R. Omega-3 fatty acids may also be denominated “n-3” or “ω-3” fatty acids. The term “omega-6 fatty acid” as used herein refers to a polyunsaturated fatty acid wherein the first double bond is located after the sixth carbon in the carbon chain (R), counting from the free methyl end of R. Omega-6 fatty acids may also be referred to as “n-6” or “ω-6” fatty acids.

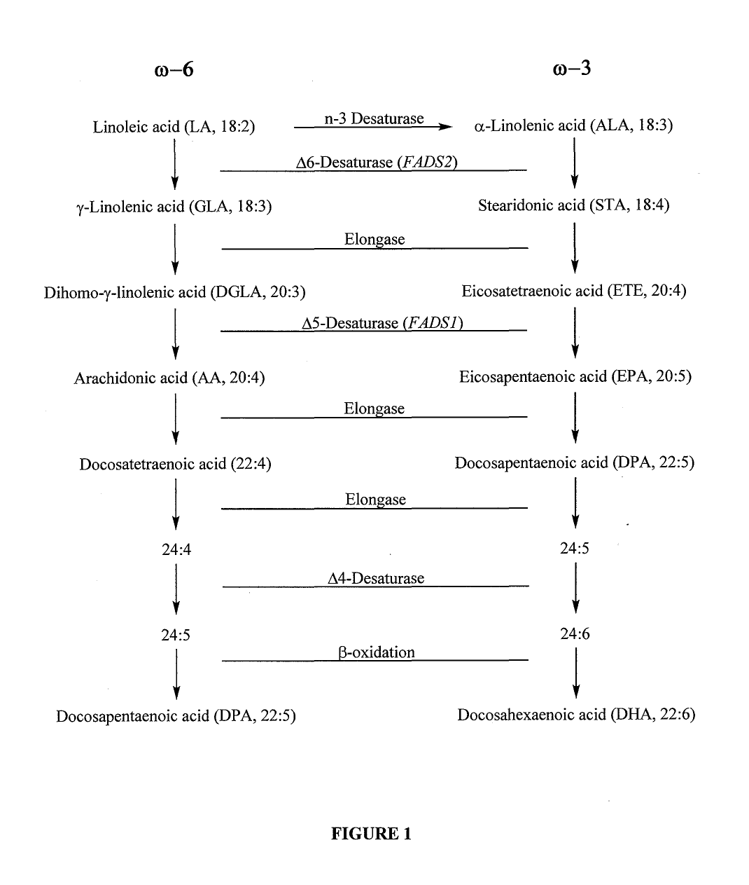

Lc-PUFAs are obtained directly from the diet and are also synthesized metabolically from certain essential mc-PUFAs. With reference to FIG. 1, the medium chain C18:2 omega-6 fatty acid linoleic acid (LA) serves as a precursor for the synthesis of the C20:4 omega-6 lc-PUFA arachidonic acid (AA), and the medium chain C18:3 omega-3 fatty acid α-linolenic acid (ALA) serves as the precursor for synthesis of the C20:5 omega-3 lc-PUFA eicosapentaenoic acid (EPA). As shown in FIG. 1, synthesis of the lc-PUFAs proceeds by elongation and desaturation steps catalyzed by specific elongase and desaturase enzymes.

As used herein, the term “efficient converter” refers to an individual who more efficiently synthesizes lc-PUFA products from mc-PUFAs precursors. Efficient converter status can be determined phenotypically, by assessing one or more measures of efficiency of enzymatic conversion, genotypically, or by determining both phenotype and genotype.

5.1.1. Determining by Phenotype

As a consequence of increased enzymatic efficiency in the biosynthetic conversion of mc-PUFAs to lc-PUFAs, efficient converters have higher ratios of lc-PUFA products to respective mc-PUFA precursors (conversely, lower ratios of mc-PUFA precursors to respective lc-PUFA products), and will at times also have higher absolute levels of lc-PUFA products than individuals who are not efficient converters. Phenotypic determination of efficient converter status can thus be performed by determining and comparing the levels of mc-PUFA precursors to respective lc-PUFA products, by determining absolute levels of lc-PUFA products, by determining and comparing the levels of omega-6 and omega-3 lc-PUFAs and/or by determining the omega-3 index. Because elongase and desaturase enzymes are shared by omega-6 and omega-3 fatty acid synthetic routes (see FIG. 1), phenotypic determination of efficient converter status can be performed by determining levels of omega-6 mc-PUFA precursors and their lc-PUFA products, omega-3 mc-PUFA precursors and their lc-PUFA products, or both. In typical embodiments, phenotypic determination is performed by measuring products and precursors in the omega-6 series. Alternatively, phenotypic determination of efficient converter status can be performed by determining and comparing levels of omega-6 and omega-3 lc-PUFA products and/or by determining the omega-3 index in red blood cells.

The rate limiting enzymes in the conversion of dietary fatty acids to AA, EPA and other lc-PUFAs are the Δ5- and Δ6-fatty acid desaturases, which are respectively encoded by fatty acid desaturase (FADS) 1 and fatty acid desaturase (FADS) 2 genes on chromosome 11q12-13 in humans (see FIG. 1). In certain embodiments, therefore, the efficient converter phenotype is conferred by more efficient activity of one or both of the Δ5- and Δ6-fatty acid desaturases.

Accordingly, in certain embodiments, efficient converter status is usefully determined by determining and comparing the levels of products to precursors wherein at least one of Δ5- and Δ6-fatty acid desaturase is required for the synthetic conversion of the measured precursor to the measured product. In some embodiments, for example, status may be determined by measuring and comparing Δ5-fatty acid desaturase product AA and its immediate Δ5-fatty acid desaturase precursor, DGLA. In certain embodiments, lc-PUFA product AA is measured and compared to the levels of precursors earlier in the biosynthetic pathway, such as GLA and/or LA. In certain embodiments, efficient converter status may usefully be determined by measuring and comparing the levels of Δ6-desaturase fatty acid product GLA and its immediate Δ6-fatty acid desaturase precursor, LA. Analogous determinations can be performed in the alternative or in addition in the omega-3 series.

In some embodiments, the measured product:precursor ratio is the ratio of AA:LA. In other embodiments, the measured product:precursor ratio is the ratio of AA:DGLA. In yet other embodiments, the measured product:precursor ratio is the ratio of AA:GLA. In various embodiments, the measured product:precursor ratio is the ratio of EPA:ALA. In some embodiments, the measured product:precursor ratio is the ratio of EPA:stearidonic acid (STA). In yet other embodiments, the measured product:precursor ratio is the ratio of EPA:eicosatetraenoic acid.

In certain embodiments, a subject is identified as an efficient converter if the product-to-precursor ratio is greater than 1. Accordingly, in some embodiments, a subject is determined to be an efficient converter if the subject's product:precursor ratio is at least about 1.5:1, at least about 2:1, at least about 2.5:1, at least about 3:1, at least about 3.5:1, at least about 4:1, at least about 4.5:1, at least about 5:1, at least about 5.5:1, at least about 6:1, at least about 6.5:1, at least about 7:1, at least about 7.5:1, at least about 8:1, at least about 8.5:1, at least about 9:1, at least about 9.5:1 at least about 10:1, at least about 11:1, at least about 12:1, at least about 13:1, at least about 14:1, or at least about 15:1. In certain embodiments, the subject is determined to be an efficient converter if the product:precursor ratio ranges between any the foregoing values, e.g., 2-6.5, 5-10, 6-8.5 or the like. In certain embodiments, a subject is identified as an efficient converter if the product:precursor ratio is at least about 6:1, at least about 6.5:1, at least about 7:1, at least about 7.5:1, at least about 8:1, at least about 8.5:1, at least about 9:1, at least about 9.5:1, at least about 10:1, at least about 11:1, at least about 12:1, at least about 12:1, at least about 13:1, at least about 14:1, or at least about 15:1.

In certain embodiments, a subject is identified as an efficient converter by measuring the absolute levels of AA in one or more tissues of the subject, such as blood, red blood cells, plasma, or serum. In various embodiments, a subject is identified as an efficient converter if AA is present in the tissues in an amount that is greater than about 5%, greater than about 6%, greater than about 7%, greater than about 8%, greater than about 9%, greater than about 10%, greater than about 11%, greater than about 12%, greater than about 13%, greater than about 14% or greater than about 15% by weight of total fatty acids in the tissues of the efficient converter. In various embodiments, a subject is determined to be an efficient converter if AA is present in the tissues of an efficient converter in an amount of about 10% or more by weight of total fatty acids in the tissues.

In various embodiments, a subject is determined to be an efficient converter by measuring the fatty acid precursor to fatty acid product ratio in the tissues of the efficient converter (“precursor:product ratio”). Thus, in some embodiments, the measured precursor:product ratio is the ratio of LA:AA. In other embodiments, the measured precursor:product ratio is the ratio of DGLA:AA. In yet other embodiments, the measured precursor:product ratio is the ratio of GLA:AA. In various embodiments, the measured precursor:product ratio is the ratio of ALA:EPA. In some embodiments, the measured precursor:product ratio is the ratio of EPA:STA. In yet other embodiments, the measured precursor:product ratio is the ratio of eicosatetraenoic acid:EPA.

In certain embodiments, a subject is identified as an efficient converter if the precursor:product ratio is less than 1. Accordingly, in some embodiments, a subject is determined to be an efficient converter if the precursor:product ratio is at least about 1:1.5, at least about 1:2, at least about 1:2.5, at least about 1:3, at least about 1:3.5, at least about 1:4, at least about 1:4.5, at least about 1:5, at least about 1:5.5, at least about 1:6, at least about 1:6.5, at least about 1:7, at least about 1:7.5, at least about 1:8, at least about 1:8.5, at least about 1:9, at least about 1:9.5, at least about 1:10, at least about 1:11, at least about 1:12, at least about 1:13, at least about 1:14, or at least about 1:15. In certain embodiments, the subject is determined to be an efficient converter if the precursor:product ratio ranges between any the foregoing values. In certain embodiments, a subject is identified as an efficient converter if the precursor:product ratio is at least about 1:6, at least about 1:6.5, at least about 1:7, at least about 1.7.5, at least about 1:8, at least about 1:8.5, at least about 1:9, at least about 1:9.5, at least about 1:10, at least about 1:11, at least about 1:12, at least about 1:13, at least about 1:14, or at least about 1:15.

In other embodiments, a subject is identified as an efficient converter by the AA:EPA ratio. In these embodiments, a subject is identified as an efficient converter if the AA:EPA ratio is greater than about 3. Thus, in certain embodiments, the subject is identified as an efficient converter if the AA:EPA ratio is at least about 3.5:1, at least about 4:1, at least about 4.5:1, at least about 5:1, at least about 5.5:1, at least about 6:1, at least about 6.5:1, at least about 7:1, at least about 7.5:1, at least about 8:1, at least about 8.5:1, at least about 9:1, at least about 9.5:1, at least about 10:1, at least about 10.5:1, at least about 11:1, at least about 11.5:1 at least about 12:1, at least about 12.5:1, at least about 13:1, at least about 13.5:1, at least about 14:1, at least about 14.5:1 or at least about 15:1. In certain embodiments, the subject is determined to be an efficient converter if the AA:EPA ratio ranges between any the foregoing values, e.g., from at least about 3:1 to at least about 3.5:1, from at least about 3:1 to at least about 8:1, from at least about 9:1 to at least about 15:1 and the like.

In other embodiments, a subject is identified as an efficient converter by the EPA:AA ratio. In these embodiments, a subject is identified as an efficient converter if the EPA:AA ratio is less than about 3. Thus, in certain embodiments, the subject is identified as an efficient converter if the EPA:AA ratio is at least about 1:3.5, at least about 1:4, at least about 1:4.5, at least about 1:5, at least about 1:5.5, at least about 1:6, at least about 1:6.5, at least about 1:7, at least about 1:7.5, at least about 1:8, at least about 1:8.5, at least about 1:9, at least about 1:9.5, at least about 1:10, at least about 1:10.5, at least about 1:11, at least about 1:11.5 at least about 1:12, at least about 1:12.5, at least about 1:13, at least about 1:13.5, at least about 1:14, at least about 1:14.5 or at least about 1:15. In certain embodiments, the subject is determined to be an efficient converter if the EPA:AA ratio ranges between any the foregoing values.

In certain embodiments, a subject is identified as an efficient converter by the omega-3 index. As used herein, the term “omega-3 index” refers to the amount of EPA and DHA in the red blood cells of a subject expressed as a percent of total fatty acids. Accordingly, in some embodiments, a subject is determined to be an efficient converter if the omega-3 index is less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5% or less than about 1% of total fatty acids. In particular embodiments, the subject is identified as an efficient converter if the omega-3 index is less than about 4% of total fatty acids. In certain embodiments, the subject is determined to be an efficient converter if the omega-3 index ranges between any of the foregoing values.

Fatty acid levels can be measured in any bodily sample, including but not limited to, a sample of whole blood, plasma, serum, membranes of red blood cells, or adipose tissue. In some embodiments, the amount of a particular fatty acid is expressed as a percentage of the total fatty acids in the sample. Fatty acid levels can be measured by any method known in the art. In certain embodiments, fatty acid levels are measured by chromatographic methods, including but not limited to, gas chromatography, liquid chromatography-mass spectrometry, gas chromatography-mass spectrometry, and high performance liquid chromatography. In other embodiments, fatty acid levels are measured by spectroscopic methods, including but not limited to, nuclear magnetic resonance and Fourier transform infrared spectroscopy.

5.1.2. Determining by Genotype

Δ5- and Δ6-fatty acid desaturases are respectively encoded by fatty acid desaturase (FADS) 1 and fatty acid desaturase (FADS) 2 genes on chromosome 11q12-13 in humans (see FIG. 1). The term “fatty acid desaturase gene” or “FADS” as used herein refers to a gene encoding a fatty acid desaturase protein in a human or non-human animal that is necessary for the synthesis of lc-PUFAs. Fatty acid desaturase genes include the human FADS genes FADS 1, which encodes the Δ5 desaturase (GenBank Accession No. NM_{—}013402.4), FADS 2, which encodes the Δ6-desaturase (GenBank Accession No. NM_{—}004265.2) and FADS 3 (GenBank Accession No. NM_{—}021727.3). Fatty acid desaturase genes and enzymes of non-human animals are readily ascertainable from GenBank (http://www.ncbi.nlm.nih.gov/genbank/).

Certain efficient converters have one or more polymorphisms in one or more fatty acid desaturase genes that lead to more efficient conversion of mc-PUFAs to lc-PUFAs. As used herein, the term “polymorphism” refers to the occurrence in a population at a rate greater than that attributable to random mutation (e.g., greater than 1%) of two or more alternate forms of a chromosomal locus that differ in nucleotide sequence or have variable numbers of nucleotide repeats. As used herein, a polymorphism “in a fatty acid desaturase gene” can be a polymorphism in the coding region of the gene, at the intron-exon boundaries, or in an upstream or downstream regulatory region of the gene.

Accordingly, in certain embodiments, efficient converter status is usefully determined genotypically, for example by determining the presence of one or more polymorphisms associated with increased efficiency of one or more desaturases.

Referring to FIG. 1, in some embodiments the polymorphism is in the FADS2 gene, which encodes the Δ6-desaturase, and results in more efficient conversion of LA to γ-linolenic acid (GLA) and/or more efficient conversion of ALA to STA. In other embodiments, the polymorphism is in the FADS1 gene, which encodes the Δ5-desaturase, and results in more efficient conversion of DGLA to AA and/or more efficient conversion of eicosatetraenoic acid to EPA.

In various embodiments, the polymorphism is a single nucleotide polymorphism (SNP). Almost all SNPs have two alleles, each allele differing in a single nucleotide. For SNPs having two alleles, the one that is more prevalent in a population is referred to as the major allele, while the less prevalent SNP is referred to as the minor allele. The presence of a SNP, as referred to herein, intends the presence of the minor allele, unless the actual genotype is specified. Thus the property of having a SNP refers to having the minor allele of the SNP, unless the actual genotype is specified.

In certain aspects, the efficient converter has one or more SNPs in a FADS gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof. In some embodiments, the efficient converter has one or more SNPs in a FADS gene selected from rs498793, rs174545, rs174548 and combinations thereof. Other single nucleotide polymorphisms found in FADS1 and FADS2 genes of humans and non-human animals can be found in the NCBI SNP database “dbSNP”, available at http://www.ncbi.nlm.nih.gov/projects/SNP/.

In various embodiments, efficient converter status is determined by detecting a polymorphism in the genome of a subject. In certain aspects, the method comprises identifying the presence of a single nucleotide polymorphism in a FADS gene selected from the group consisting of rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof. Polymorphisms, including single nucleotide polymorphisms, can be detected in a sample, e.g., a sample containing nucleated blood cells, by any method known in the art. Methods of detecting SNPs include DNA sequencing, methods that require allele specific hybridization of primers or probes (e.g., dynamic allele-specific hybridization (DASH), use of molecular beacons, and SNP microarrays such as the Affymetrix Human SNP Array 6.0), allele specific incorporation of nucleotides to primers bound close to or adjacent to the polymorphisms (“single base extension”, or “minisequencing”), allele-specific ligation of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation may also be employed where the SNP lies in a coding region and results in an amino acid change.

5.1.3. Determining by Phenotype and Genotype

In certain embodiments, a subject is identified as an efficient converter by both phenotypic and genotypic determination, as above described.

In certain embodiments, a subject is identified as an efficient converter by determining a ratio of AA:DGLA and/or measuring the level of AA in the body of the subject and by detecting the presence of a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof. In particular embodiments, the AA:DGLA ratio is greater than about 6 and/or the AA level is greater than about 10% by weight of total fatty acids in the sample.

In other embodiments, a subject is identified as an efficient converter by determining a ratio of AA:EPA and by detecting the presence of a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof. In certain embodiments, the AA:EPA ratio is greater than about 3.

In further embodiments, a subject is identified as an efficient converter by determining the omega-3 index and by detecting the presence of a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof. In particular embodiments, the subject is identified as an efficient converter if the omega-3 index is less than about 4% of total fatty acids.

5.2. Methods of Identifying a Subject Susceptible to ADHD

The inventors have discovered that a subject susceptible to developing ADHD can be identified by determining whether the subject is an efficient converter of mc-PUFA to lc-PUFA, wherein efficient converter status indicates susceptibility to ADHD.

The term “subject” includes humans and non-human animals.

The term “attention deficit hyperactivity disorder” or “ADHD” refers to a syndrome of inattention, hyperactivity, and impulsivity and includes three subtypes: (i) predominantly inattentive (formerly known as attention deficit disorder or ADD), (ii) predominantly hyperactive-impulsive, and (iii) combined inattentive and hyperactive-impulsive. Unless otherwise noted, the term “attention deficit hyperactivity disorder” or “ADHD” refers to all subtypes of the disorder. Diagnosis is made by clinical criteria and is based on comprehensive medical, developmental, educational, and psychological evaluations.

In certain embodiments, the method for determining whether the subject is an efficient converter of mc-PUFA to lc-PUFA is selected from those above-described. In various embodiments, a subject susceptible to ADHD is identified by determining efficient converter status and by psychiatric assessment. In some embodiments, the psychiatric assessment is based on the diagnostic criteria set forth in the Diagnostic and Statistical Manual of Mental Disorders (DSM-IV-TR). In other embodiments, the psychiatric assessment is based on the diagnostic criteria set forth in the International Statistical Classification of Diseases and Related Health Problems, 10th Revision (ICD-10). In particular embodiments, a subject who is susceptible to ADHD does not meet all of the DSM-IV-TR or ICD-10 diagnostic criteria for ADHD, but evidences one or more of the signs listed therein.

5.3. Methods of Treating ADHD

The inventors have further discovered that ADHD can be treated or prevented in such efficient converters by administering an effective amount of compositions comprising omega-3 lc-PUFAs.

Thus, methods are provided herein for treating or preventing ADHD in a subject who is an efficient converter of mc-PUFAs to lc-PUFAs. The methods comprise administering to a subject who has been determined to be an efficient converter of mc-PUFAs to lc-PUFAs an amount of a composition comprising omega-3 lc-PUFAs effective to treat or prevent ADHD.

In typical embodiments, subjects are determined to be efficient converters according to the methods above-described.

5.3.1. Omega-3 lc-PUFA Compositions

The methods described herein for treating or preventing ADHD in efficient converters of mc-PUFAs to lc-PUFAs comprise administering to a subject who has been determined to be an efficient converter of mc-PUFAs to lc-PUFAs an amount of a composition comprising omega-3 lc-PUFAs effective to treat or prevent ADHD.

In certain embodiments, the composition comprises the lc-PUFA all-cis-5,8,11,14,17-eicosapentaenoic acid (EPA, C20:5). In other embodiments, the composition comprises EPA and all-cis-4,7,10,13,16,19-docosahexaenoic acid (DHA, C22:6). In certain embodiments the composition comprises fatty acids in the form of a pharmaceutically acceptable ester, such as a C1-C5 alkyl ester, e.g., methyl ester, ethyl ester, propyl ester, butyl ester and the like. In particular embodiments, the fatty acids in the composition are in the form of an ethyl ester. In other particular embodiments, the fatty acids in the composition are in the free acid form. In still other embodiments, the fatty acids in the composition are in the salt form. Unless otherwise noted, “EPA,” “DHA,” and the like are meant to encompass free acid forms, pharmaceutically acceptable esters, amides, triglycerides, diglycerides, monoglycerides, phospholipids, derivatives including, but not limited to, alpha-substituted derivatives, conjugates, including, but not limited to, conjugates with active ingredients such as salicylates, fibrates, niacin, cyclooxygenase inhibitors, or antibiotics, or salts thereof, or mixtures of any of the foregoing.

The fatty acid content of the compositions described herein can be determined by any method known in the art. Exemplary methods for determining the fatty acid profile of a composition include, but are not limited to, chromatographic methods such as gas chromatography (GC), gas liquid chromatography (GLC), mass spectrometry (MS), high performance liquid chromatography (HPLC), reverse phase HPLC, thin layer chromatography (TLC), GC-MS and TLC-GLC, and the like, and spectroscopic methods such as nuclear magnetic resonance spectroscopy (NMR) and Fourier transform infrared spectroscopy (FTIR).

In various embodiments, the composition comprises EPA in an amount of at least about 20%, at least about 25%, at least about 30%, at least about 35%, at least about 40%, at least about 45%, at least about 50%, at least about 55%, of at least about 60%, at least about 65%, at least about 70%, at least about 75%, at least about 80%, at least about 85%, at least about 90%, at least about 95%, at least about 96%, at least about 97%, at least about 98%, at least about 99% or at least about 100% by weight of total fatty acids in the composition. In certain embodiments, the composition comprises EPA in an amount ranging between any of the foregoing values, e.g., 20%-75% by weight, 40%-50% by weight, 50%-55% by weight, 50%-60% by weight, 75%-85% by weight, 90%-98% by weight, 30%-100% by weight or the like, of total fatty acids in the composition.

In certain embodiments, the composition comprises DHA in an amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or of at least about 95% by weight of total fatty acids in the composition. In certain embodiments, the composition comprises DHA in an amount ranging between any of the foregoing values, e.g., 15%-25% by weight, 15%-30% by weight, 30%-45% by weight, 50%-85% by weight, or the like, of total fatty acids in the composition.

In certain embodiments, the composition comprises DHA in an amount of not more than about 10%, not more than about 9%, not more than about 8%, not more than about 7%, not more than about 6%, not more than about 5%, not more than about 4%, not more than about 3%, not more than about 2%, not more than about 1%, or not more than about 0.5% of total fatty acids in the composition. In a particular embodiment, the composition comprises no detectable DHA. In another particular embodiment, the composition comprises EPA in the ethyl ester form in an amount of at least about 96% by weight of total fatty acids and no detectable DHA.

In certain embodiments, the composition comprises EPA in an amount of at least about 20%, at least about 25%, at least about 30%, at least about 35%, at least about 40%, at least about 45%, at least about 50%, at least about 55%, of at least about 60%, at least about 65%, at least about 70%, at least about 75%, at least about 80%, at least about 85%, at least about 90%, at least about 95%, at least about 96%, at least about 97%, at least about 98%, at least about 99% or of at least about 100% by weight of total fatty acids in the composition, and DHA in an amount of at least about 5%, at least about 10%, at least about 15%, at least about 20%, at least about 30%, at least about 40%, at least about 50%, at least about 55%, of at least about 60%, at least about 65%, at least about 70%, at least about 75%, at least about 80%, at least about 85%, at least about 90% or of at least about 95% by weight of total fatty acids in the composition. In certain embodiments, the composition comprises EPA in an amount ranging between any of the foregoing values, e.g., 20%-75% by weight, 30%-100% by weight, 40%-50% by weight, 50%-55% by weight, 50%-60% by weight, 75%-85% by weight, 90%-95% by weight, or the like, of total fatty acids in the composition and DHA in an amount ranging between any of the foregoing values, e.g., 10%-30% by weight, 15%-25% by weight, 15%-30% by weight, 30%-45% by weight, 50%-55% by weight, 50%-60% by weight, or the like, of total fatty acids in the composition.

In various embodiments, the composition comprises EPA in an amount of at least about 1 g, at least about 1.5 g, at least about 2 g, at least about 2.5 g, at least about 3 g, at least about 3.5 g, or at least about 4 g. In some embodiments, the composition comprises DHA in an amount of at least about 1 g, at least about 1.5 g, at least about 2 g, at least about 2.5 g, at least about 3 g, at least about 3.5 g or at least about 4 g. In certain embodiments, the composition comprises EPA or DHA in an amount ranging between any of the foregoing values, e.g., 1 g-3 g, 2.5 g-4 g, 1.5 g-3.5 g and the like. In some embodiments, the composition comprises EPA and DHA in a combined amount of at least about 1 g, at least about 1.5 g, at least about 2 g, at least about 2.5 g, at least about 3 g, at least about 3.5 g or at least about 4 g. In certain embodiments, the composition comprises EPA and DHA in a combined amount ranging between any of the foregoing values, e.g., 1 g-3.5 g, 2 g-4 g, 1.5 g-3 g and the like.

In certain embodiments, the composition comprises EPA and DHA in a weight ratio of about 1:1, of about 1.25:1, of about 1.5:1, of about 1.75:1, of about 2:1, of about 2.25:1, of about 2.5:1, of about 2.75:1, of about 3:1, of about 3.25:1, of about 3.5:1, of about 3.75:1, of about 4:1, of about 4.25:1, of about 4.5:1, of about 4.75:1 or of about 5:1. In particular embodiments, the composition comprises EPA and DHA in a weight ratio of about 2:1, of about 3:1, of about 1.24:1, of about 4:1 or of about 4.1:1. In a particular embodiment, the composition comprises EPA and DHA in the ethyl ester form in a weight ratio of about 1.24:1 to about 1.43. In certain embodiments, the composition comprises EPA and DHA in the free acid form in weight ratios ranging from about 2:1 to about 4:1.

In some embodiments, the composition comprises EPA in the ethyl ester form in an amount of from about 40% to about 50% by weight, and DHA in the ethyl ester form in an amount of from about 30% to about 45% by weight of total fatty acids in the composition. In other embodiments, the composition comprises EPA in the ethyl ester form in an amount of from about 43% to about 49.5% by weight, and DHA in the ethyl ester form in an amount of from about 34.7% to about 40.3% by weight of total fatty acids in the composition. In other embodiments, the composition comprises EPA ethyl ester in an amount of from about 70% to about 80% by weight, and DHA in an amount of from about 10% to about 20% by weight. In still other embodiments, the pharmaceutical composition comprises EPA in the ethyl ester form in an amount of at least about 96% by weight, and no detectable DHA. In particularly preferred embodiments, the composition comprises EPA in the free acid form in an amount of from about 50% to about 60% by weight, and DHA in the free acid form in an amount of from about 15% to about 25% by weight of total fatty acids in the composition.

In various embodiments, the composition comprises an omega-3 or omega-6 fatty acid other than EPA or DHA in an amount of not more than about 30%, not more than about 25%, not more than about 20%, not more than about 15%, not more than about 10%, not more than about 9%, not more than about 8%, not more than about 7%, not more than about 6%, not more than about 5%, not more than about 4%, not more than about 3%, not more than about 2%, or not more than about 1%, by weight of the total weight of fatty acids in the composition. In certain embodiments, the composition comprises an omega-3 or omega-6 fatty acid other than EPA or DHA in an amount of from about 12% to about 20% by weight of total weight of fatty acids in the composition. Illustrative examples of an “omega-3 or omega-6 fatty acid other than EPA and DHA” include, but are not limited to, saturated fatty acids, mono-unsaturated fatty acids, omega-6 fatty acids such as arachidonic acid (AA, C20:4), linoleic acid (LA, C18:2), γ-linolenic acid (GLA, C20:3), and α-linolenic acid (ALA, C18:3), and omega-3 fatty acids such as stearidonic acid (STA, C18:4), eicosatrienoic acid (ETA, C20:3), eicosatetraenoic acid (ETE, C20:4), docosapentaenoic acid (DPA, C22:5), heneicosapentaenoic acid (HPA, C21:5), tetracosapentaenoic acid (C24:5) and tetracosahexaenoic acid (C24:6).

In certain embodiments, the omega-3 or omega-6 fatty acid other than EPA or DHA is in the ester form. In other embodiments, the omega-3 or omega-6 fatty acid other than EPA or DHA is in the free acid form. In particular embodiments, the composition comprises DPA, STA, HPA, ETE and ALA in the ethyl ester form in a combined total amount of from about 12% to about 20% by weight of total weight of fatty acids in the composition.

In certain embodiments, the composition comprises no detectable omega-3 fatty acids other than EPA and DHA. In various embodiments, the composition comprises omega-3 fatty acids other than DHA and EPA in an amount of not more than about 1% by weight, not more than about 2% by weight, not more than about 3% by weight, not more than about 4% by weight, not more than about 5% by weight, not more than about 6% by weight, not more than about 7% by weight, not more than about 8% by weight, not more than about 9% by weight, not more than about 10% by weight, not more than 11% by weight, not more than 12% by weight, not more than about 13% by weight, not more than about 14% by weight or not more than about 15% by weight, not more than about 16% by weight, not more than about 17% by weight, not more than about 18% by weight, not more than about 19% or not more than about 20% by weight of total fatty acids in the composition. In certain embodiments, the composition comprises omega-3 fatty acids other than EPA and DHA in an amount ranging between any of the foregoing values, e.g., 1%-15% by weight, 4%-12% by weight, 10%-15% by weight, 5%-10% by weight, 1%-4% by weight, and the like, of total fatty acids.

In certain embodiments, the composition comprises total omega-6 fatty in a combined amount of not more than about 20% by weight, not more than about 19% by weight, not more than about 18% by weight, not more than about 17% by weight, not more than about 16% by weight, not more than about 15% by weight, not more than about 14% by weight, not more than about 13% by weight, not more than about 12% by weight, not more than about 11% by weight, not more than about 10% by weight, not more than about 9% by weight, not more than about 8% by weight, not more than about 7% by weight, not more than about 6% by weight, not more than about 5% by weight, not more than about 4% by weight, not more than about 3% by weight, not more than about 2% by weight, not more than about 1% by weight, or not more than about 0.5% by weight of total fatty acids in the composition. In some embodiments, the composition comprises omega-6 fatty acids in a combined amount of not more than about 10% by weight of total fatty acids in the composition. In some embodiments, the composition comprises omega-6 fatty acids in a combined amount of not more than about 10% by chromatographic area, of total fatty acids in the composition.

In some embodiments, the composition comprises AA in an amount of not more than about 10% by weight, not more than about 9% by weight, not more than about 8% by weight, not more than about 7% by weight, not more than about 6% by weight, not more than about 5.5% by weight, not more than about 5% by weight, not more than about 4.5% by weight, not more than about 4% by weight, not more than about 3.5% by weight, not more than about 3% by weight, not more than about 2.5% by weight, not more than about 2% by weight, not more than about 1.5% by weight, not more than about 1% by weight or not more than about 0.5% by weight of total fatty acids in the composition. In certain embodiments, the composition comprises AA in an amount of not more than about 4.5% by weight of total fatty acids in the composition. In some embodiments, the composition comprises AA in an amount of not more than about 4.5% by chromatographic area of total fatty acids in the composition.

In various embodiments, the composition comprises other fatty acids, such as saturated fatty acids in an amount of not more than about 5% by weight, not more than about 4% by weight, not more than about 3% by weight, not more than about 2% by weight or not more than about 1% by weight of total fatty acids in the composition, and/or monounsaturated fatty acids in an amount of not more than about 7% by weight, not more than about 6% by weight, not more than about 5% by weight, not more than about 4% by weight, not more than about 3% by weight, not more than about 2% by weight or not more than about 1% by weight of total fatty acids in the composition. In some embodiments, the composition comprises unsaturated fatty acids other than polyunsaturated fatty acids and monounsaturated fatty acids in an amount of not more than about 7% by weight, not more than about 6% by weight, not more than about 5% by weight, not more than about 4% by weight, not more than about 3% by weight, not more than about 2% by weight, or not more than about 1% by weight of total fatty acids in the composition. In a particular embodiment, the composition comprises saturated fatty acids in an amount of not more than about 3% by weight, monounsaturated fatty acids in an amount of not more than 5% by weight, and unsaturated fatty acids other than omega-3 and omega-6 polyunsaturated fatty acids and monounsaturated fatty acids in an amount of not more than 5% by weight of total fatty acids in the composition. In another particular embodiment, the composition comprises saturated fatty acids in an amount of not more than about 3%, monounsaturated fatty acids in an amount of not more than 5%, and unsaturated fatty acids other than omega-3 and omega-6 polyunsaturated fatty acids and monounsaturated fatty acids in an amount of not more than 5% by chromatographic area of total fatty acids in the composition.

In a particular embodiment, the fatty acid composition for use in the methods described herein comprises from about 50% to about 60% by weight of EPA in the free acid form, from about 15% to about 25% by weight of DHA in the free acid form, from about 0% to about 15% by weight of omega-3 fatty acids other than EPA and DHA. In certain preferred embodiments, the fatty acid composition comprises from about 70% by weight to about 80% by weight of EPA and DHA, and from about 80% by weight to about 95% by weight of omega-3 fatty acids, including EPA and DHA. In particular embodiments, the composition further comprises AA in an amount of not more than about 4.5% by weight, total omega-6 fatty acids in an amount of not more than about 10% by weight, saturated fatty acids in an amount of not more than about 3% by weight, monounsaturated fatty acids in an amount of not more than about 5% by weight and unsaturated fatty acids other than omega-3 and omega-6 polyunsaturated fatty acids and monounsaturated fatty acids in an amount of not more than 5% by weight of total fatty acids in the composition. In other embodiments, the composition further comprises AA in an amount of not more than about 4.5%, total omega-6 fatty acids in an amount of not more than about 10%, saturated fatty acids in an amount of not more than about 3%, monounsaturated fatty acids in an amount of not more than about 5% and unsaturated fatty acids other than omega-3 and omega-6 polyunsaturated fatty acids and monounsaturated fatty acids in an amount of not more than 5% by chromatographic area of total fatty acids in the composition.

The sources of the fatty acids for use in the pharmaceutical compositions described herein include, but are not limited to, fish oil, marine microalgae oils, plant oils or combinations thereof. In some embodiments, the fatty acids are derived from algae. In a particular embodiment, the source of the fatty acids for use in the pharmaceutical compositions described herein is fish oil. Because the fatty acids are derived from natural sources, in certain embodiments, the compositions include trace amounts of other substances derived from the source oil, such as fat soluble vitamins, e.g., vitamin A and/or vitamin D, and/or cholesterol.

The fatty acids for use in the compositions described herein can be isolated and purified by any method known in the art. In certain embodiments, where a composition of fatty acid esters is desired, the fatty acids are extracted and purified from marine oils by (i) refining and deodorizing crude marine oil triglycerides; (ii) esterifying the fatty acids; (iii) fractionating and concentrating the esters, e.g., by fractional distillation; (iv) removing saturated fatty acids and other contaminants; and (v) concentrating the fatty acid esters, e.g., by distillation, to achieve the final product. In certain embodiments, where a composition of free fatty acids is desired, the fatty acid esters obtained after step (iv) can be hydrolyzed, for example, by base hydrolysis, and then be further purified by fractional distillation. In other embodiments, the marine oil can be deacidified before the refining step by, for example, distillation or washing with sodium hydroxide, to remove the free fatty acids. Exemplary methods of obtaining fatty acid compositions are found, for example, in U.S. Pat. Nos. 5,656,667 and 6,630,188 to Norsk Hydro AS, U.S. Pat. No. 7,807,848 to Ocean Nutrition Canada, Ltd. And U.S. Pat. No. 7,119,118 to Laxdale Ltd.

In certain embodiments, the compositions of the disclosure contain one or more pharmaceutically acceptable carriers, excipients or stabilizers (referred to as “excipients” herein) typically employed in the art, i.e., fillers, stabilizers, extenders, binders, humidifiers, surfactants, lubricants, preservatives, antioxidants, flavorants, colorants and other miscellaneous additives. Specific examples of pharmaceutically acceptable carriers and excipients that can be used to formulate oral dosage forms are described in the Handbook of Pharmaceutical Excipients, American Pharmaceutical Association (1986) and in Remington's Pharmaceutical Sciences, 16th edition (Osol, ed. 1980). Such additives must be nontoxic to the recipients at the dosages and concentrations employed. An excipient can be inert or it can possess pharmaceutical benefits.

In certain embodiments, the omega-3 compositions described herein comprise an antioxidant. Suitable antioxidants include, but are not limited to, tocopherols, such as α-tocopherol, β-tocopherol, γ-tocopherol, δ-tocopherol, and tocotrienols, such as α-tocotrienol, β-tocotrienol, γ-tocotrienol and δ-tocotrienol. In certain embodiments, an antioxidant can be present in the composition in an amount of from about 0.1% to about 0.5% by weight, of from about 0.15% to about 0.25% by weight, of from about 0.2% to about 0.4% by weight, or of from about 0.25% to about 0.35% of total fatty acids in the composition. In one embodiment, the antioxidant is α-tocopherol that is present in an amount of from about 0.4% to about 0.44% by weight of the composition. In another embodiment, the α-tocopherol is present in the composition in an amount of about 0.27% to about 0.33% by weight of the composition.

Excipients are selected with respect to the intended form of administration and consistent with conventional pharmaceutical practices. Preferably, the compositions are administered orally, e.g., in tablets, capsules, powders, syrups, suspensions, and the like. In particular embodiments, the pharmaceutical dosage form is a capsule. In certain embodiments, the dosage form is a hard gelatin capsule. In other embodiments, the dosage form is a soft gelatin capsule. A gelatin capsule for encapsulating the pharmaceutical compositions described herein can be made from Type A gelatin, gelatin extracted by a process comprising an acid pre-treatment of a collagen source, e.g., pig skin, or from Type B gelatin, gelatin extracted by a process comprising an alkaline pre-treatment of a collagen source. Sources of collagen for the production of gelatin include, but are not limited to, cows, pigs and fish. Capsules can also be made from substances that are not animal by-products such as agar-agar, carrageenan, pectin, konjak, guar gum, food starch, modified corn starch, potato starch, and tapioca. Non-animal sources of materials that can be used to make capsules are described in U.S. Patent Publication No. 2011/0117180, assigned to Ocean Nutrition Canada Ltd. In a particular embodiment, the dosage form of the pharmaceutical compositions described herein is a soft gelatin capsule made from Type A porcine gelatin.

In addition to gelatin or a non-animal gelling agent, in particular embodiments, a soft gelatin capsule shell contains a plasticizer and water. Plasticizers for use in soft gelatin capsules include, but are not limited to, small polyhydroxy compounds such as glycerol, sorbitol, propylene glycol, sucrose, maltitol and mixtures thereof. In certain embodiments, the gelatin capsule contains one or more substances selected from a preservative such as methyl paraben or propylmethyl paraben, a colorant, an opacifying agent such as titanium dioxide, a flavoring agent, a sugar, a chelating agent and a medicament. In certain embodiments, the gelatin capsule comprises water in an amount of at least about 1% by weight, of at least about 2% by weight, of at least about 3% by weight, of at least about 4% by weight, of at least about 5% by weight, of at least about 6% by weight, of at least about 7% by weight, of at least about 8% by weight, of at least about 9% by weight or of at least about 10% by weight of the composition. In certain embodiments, the gelatin capsule comprises water in an amount ranging between any of the foregoing values, e.g., 1%-5% by weight, 2%-8% by weight, 6%-10% by weight, 5%-10% by weight, and the like. In a particular embodiment, the gelatin capsule comprises water in an amount of between about 6% and about 10% by weight of the composition. In some embodiments, the gelatin capsule comprises a plasticizer in an amount of not more than about 0.1%, of not more than about 0.2%, of not more than about 0.3%, of not more than about 0.4%, of not more than about 0.5%, of not more than about 0.6%, of not more than about 0.7%, of not more than about 0.8%, of not more than about 0.9% or of not more than about 1% by weight of the composition.

In certain embodiments, the gelatin capsule is uncoated. In other embodiments, the gelatin capsule is coated to delay release of the fatty acid composition until after passage through the stomach. In certain embodiments, release of the fatty acid composition is delayed for at least 30 minutes after ingestion. In other embodiments, release of the fatty acid composition is delayed for from about 30 minutes to about 60 minutes after ingestion. Suitable coatings for achieving delayed release of the fatty acid composition are known to one of skill in the art and include enteric coatings that are resistant to dissolution in a time dependent and/or pH dependent manner. In a particular embodiment, the gelatin capsule is coated with an enteric coating that releases the fatty acid composition in a time dependent manner. In various embodiments, the coating is selected from cellulose acetate trimellitate, cellulose acetate phthalate and poly(ethylacrylate-methylacrylate). In one embodiment, the dosage form is an enteric coated soft gelatin capsule and the enteric coating used for time dependent dissolution is a neutral polyacrylate such as poly(ethylacrylate-methylmethacrylate), such as Eudragit N E 30-D (Rohm Pharma GmbH), which has an average molecular weight of about 800,000. In a particular embodiment, the dosage form is a coated soft gelatin capsule as described in U.S. Pat. No. 7,960,370 to Tillotts Pharma AG.

In some embodiments, the dosage form is selected from a 250-mg dosage form, a 300-mg dosage form, a 350-mg dosage form, a 400-mg dosage form, a 450-mg dosage form, a 500-mg dosage form, a 600-mg dosage form, a 700-mg dosage form, an 800-mg dosage form, a 900-mg dosage form, a 1-g dosage form, a 1.2-g dosage form and a 1.5-g dosage form. In some embodiments, the dosage form is a 1.5-g dosage form. In particular embodiments, the dosage form is a 1-g dosage form. In certain embodiments, the 1-g dosage form is an enteric coated soft Type A gelatin capsule as described above. In certain embodiments, the 1-g dosage form comprises total omega-3 fatty acids in an amount of at least about 800 mg, of at least about 825 mg, of at least about 850 mg, of at least about 875 mg, of at least about 900 mg, of at least about 925 mg, of at least about 950 mg, of at least about 960 mg, or of at least about 975 mg per 1-g dosage form. In certain embodiments, the 1-g dosage form comprises total omega-3 fatty acids in an amount ranging between any of the foregoing values, e.g., 800 mg-950 mg, 875 mg-900 mg, 900 mg-975 mg, and the like. In one particular embodiment, the dosage form is a 1-g soft gelatin capsule that comprises at least about 900 mg of the ethyl esters of total omega-3 fatty acids. In another particular embodiment, the dosage form is a 1-g soft gelatin capsule that comprises from about 800 mg to about 950 mg of total omega-3 fatty acids in the free acid form. In yet another embodiment, the dosage form is a 500-mg capsule that comprises from about 400 mg to about 495 mg, from about 425 mg to about 480 mg, or from about 450 mg to about 490 mg of the ethyl ester form of EPA. In still other embodiments the dosage form is a 1.5-g capsule that comprises at least about 1,300 mg, at least about 1,350 mg, at least about 1,400 mg, or at least about 1,450 mg of EPA and DHA ethyl esters.

5.3.2. Dose of Composition Comprising Omega-3 lc-PUFA

Dosage, dosage forms, and dosage schedule of omega-3 lc-PUFAs suitable for treatment or prevention of ADHD are described below with respect to methods of treating ADHD.

5.4. Methods of Treating with ADHD Therapy

Methods are also provided for treating or preventing ADHD in subjects in need thereof, comprising (a) determining whether the subject is an efficient converter of mc-PUFA to lc-PUFA and (b) in those subjects determined to be efficient converters of mc-PUFA to lc-PUFA, administering an amount of a composition comprising omega-3 lc-PUFAs effective to treat or prevent ADHD concomitantly with administering an effective amount of an ADHD therapy.

In typical embodiments, the method for determining whether the subject is an efficient converter of mc-PUFA to lc-PUFA is selected from those above-described. In those subjects determined to be efficient converters of mc-PUFA to lc-PUFA, the methods comprise administering an amount of a composition comprising omega-3 lc-PUFAs effective to treat or prevent ADHD. In typical embodiments, the compositions comprising omega-3 lc-PUFAs are selected from those above-described.

The composition comprising omega-3 lc-PUFAs is administered concomitantly with an effective amount of an ADHD therapy.

The term “ADHD therapy” as used herein refers to administration of one or more agents that is indicated for reducing the symptoms of ADHD and improving functioning in a subject diagnosed with ADHD and/or to psychosocial therapies.

In certain embodiments, exemplary ADHD therapies are medicinal and include agents selected from the group consisting of a stimulant such as amphetamine (Adderall®, Adderall XR®), methylphenidate (Concerta®, Daytrana®, Metadate ER®, Metadate CD®, Methylin®, Ritalin®, Ritalin SR®, Ritalin LA®), methamphetamine hydrochloride (Dexoxyn®), dextroamphetamine (Dexedrine®, Dextrostat®, Spansule®, ProCentra®), dexmethylphenidate (Focalin®, Focalin XR®) and lisdexamfetamine dimesylate (Vyvanse®), a selective norepinephrine reuptake inhibitor such as atomoxetine (Strattera®, Tomoxetin® Attentin®), amantidine, modafinil (Provigil®), an antidepressant such as bupropion (Wellbutrin®), venlafaxine (Effexor®), milnacipran (Savella), reboxetine (Edronax®), desipramine or nortriptyline, an α-2 norepinephrine receptor agonists such as clonidine or guanfacine (Intuniv®, Tenex), and combinations thereof.

In other embodiments, exemplary ADHD therapies are psychosocial and include psychoeducational input, behavior therapy, cognitive behavioral therapy, interpersonal psychotherapy (IPT), family therapy, school-based interventions, social skills training and parent management training. In particular embodiments, ADHD therapies include a combination of medicinal and psychosocial therapies.

In certain embodiments, the subject who suffers from or is susceptible to ADHD also suffers from one or more disorders that co-exist with ADHD. In particular embodiments, the one or more co-existing disorders is selected from the group consisting of oppositional defiant disorder, conduct disorder, antisocial personality disorder, borderline personality disorder, primary disorder of vigilance, a mood disorder, bipolar disorder, anxiety disorder, obsessive compulsive disorder, Tourette syndrome, a learning disorder and substance abuse.

The methods comprise administering an amount of a composition comprising omega-3 lc-PUFAs effective to treat ADHD. The effective amount may be prior determined or, in certain embodiments, the methods further comprise measuring the efficacy of an ADHD therapy, e.g., to determine whether the dosage of omega-3 lc-PUFAs is sufficient to treat ADHD. Optionally, the methods comprise adjusting dosage of the ADHD therapy, and/or the dose of the composition comprising omega-3 lc-PUFAs, based on the measured efficacy of the ADHD therapy.

The effect of an ADHD therapy and/or the methods and compositions described herein on ADHD can be determined by any method known in the art. In certain embodiments, the progression of ADHD can be monitored using the diagnostic criteria set forth in the DSM-IV-TR. In other embodiments, the progression of ADHD can be monitored using the diagnostic criteria set forth in the ICD-10. In still other embodiments, the progression of ADHD can be monitored using multiple criteria, for example, the explicit diagnostic criteria set forth in the DSM-IV-TR together with information about the subject's signs, symptoms and/or behavior in more than one setting, and information about coexisting conditions.

The amount of an omega-3 lc-PUFA composition that is effective for the treatment and/or prevention of ADHD can be determined by standard clinical techniques. In addition, in vitro and/or in vivo assays can optionally be employed to help identify optimal dosage ranges. The precise dose to be employed will also depend on, e.g., the route of administration and the seriousness of the condition, and can be decided according to the judgment of a practitioner and/or each subject's circumstances. In other examples thereof, variations will necessarily occur depending upon the age, weight and physical condition (e.g., hepatic and renal function) of the subject, the severity of the symptoms, the frequency of the dosage interval, the presence of any deleterious side-effects, the presence of co-existing conditions, and the particular fatty acid compositions and/or ADHD therapy utilized, among other things.

Suitable effective doses of the omega-3 lc-PUFA compositions for use in the methods described herein range from about 1 g to about 10 g per day, from about 2 g to about 9 g per day, from about 3 g to about 8 g per day, from about 4 g to about 7 g per day, from about 5 g to about 6 g per day, depending upon body size and the seriousness of the condition to be treated. In a particular embodiment, the effective dosage amounts of the omega-3 lc-PUFA compositions range from about 1 g to about 4 g per day. Accordingly, a suitable effective dose of the omega-3 lc-PUFA compositions described herein is at least about 1 g/day, at least about 2 g/day, at least about 3 g/day, at least about 4 g/day, at least about 5 g/day, at least about 6 g/day, at least about 7 g/day, at least about 8 g/day, at least about 9 g/day or at least about 10 g/day.

In certain embodiments, the omega-3 lc-PUFA compositions for use in the methods described herein comprise EPA. In some embodiments, the omega-3 lc-PUFA compositions comprise DHA. Suitable effective doses of EPA or DHA for use in the methods described herein range from about 1 g to about 10 g per day, from about 2 g to about 9 g per day, from about 3 g to about 8 g per day, from about 4 g to about 7 g per day, from about 5 g to about 6 g per day, depending upon body size and the seriousness of the condition to be treated. In a particular embodiment, the effective dosage amounts of EPA or DHA range from about 1 g to about 4 g per day. In various embodiments, the omega-3 lc-PUFA compositions for use in the methods described herein comprise EPA and DHA. Suitable effective doses of EPA and DHA for use in the methods described herein range from a combined amount of about 1 g to about 10 g per day, from about 2 g to about 9 g per day, from about 3 g to about 8 g per day, from about 4 g to about 7 g per day, from about 5 g to about 6 g per day, depending upon body size and the seriousness of the condition to be treated. In a particular embodiment, the effective dosage amounts of EPA and DHA range from a combined amount of about 1 g to about 4 g per day.

The effective doses of omega-3 lc-PUFA compositions described herein refer to total amounts administered. An effective dose can be administered in a single dose or as a divided dose. In one embodiment, an effective dose is administered once about every 24 h. In another embodiment, an effective dose is administered in 2 doses over the course of 24 h. An effective dose may be in a single dosage form, e.g., in one capsule or tablet, or divided into multiple dosage forms, e.g., 2, 3 or 4 capsules. In a particular embodiment, an effective dose is administered once per day, at or near the same time every day. In another particular embodiment, the effective dose is administered in two doses over 24 h.

The composition comprising omega-3 lc-PUFAs effective to treat ADHD is administered concomitantly, or adjunctively, with administering an effective amount of an ADHD therapy.

By “concomitant” or “adjunctive” administration, it is intended that the composition comprising omega-3 lc-PUFAs be administered at and for a time sufficient to ensure the presence of effective levels of PUFAs concurrently with the presence in the blood of the ADHD therapy. Thus, the omega-3 lc-PUFA composition can be administered concurrently with administration of the ADHD therapy, and may be started before, and/or continued after, cessation of ADHD therapy.

In some embodiments of the methods described herein, in which a medicinal ADHD therapy is provided, the omega-3 lc-PUFA composition can be administered in advance of the ADHD therapy, e.g., as a loading dose. In some embodiments, the omega-3 lc-PUFA composition is administered for at least 1 day, for at least 2 days, for at least 3 days, for at least 4 days, for at least 5 days, for at least 6 days, for at least 1 week, for at least 2 weeks, for at least 3 weeks, or for at least 1 month or more in advance of the ADHD therapy. In other embodiments, the omega-3 lc-PUFA composition is first administered in advance of the ADHD therapy, and is then administered concurrently with the ADHD therapy. In still other embodiments, the omega-3 lc-PUFA composition is administered concurrently with the ADHD therapy for the duration of the ADHD therapy.

In certain embodiments, an effective dose can be administered for 2 weeks, 4 weeks, 6 weeks, 8 weeks, 12 weeks, 4 months, 6 months, 8 months, 12 months, 24 months or longer, depending on the nature and severity of the condition. In certain embodiments, an effective dose is administered daily during the life of the subject. In certain embodiments, where the omega-3 lc-PUFA composition is administered adjunctively with a medicinal ADHD therapy, the omega-3 lc-PUFA composition is administered for the duration of the ADHD therapy.

In certain embodiments, the omega-3 compositions are administered with food. In other embodiments, the compositions are administered to a subject who is fasting. In particular embodiments, the omega-3 compositions are administered to a subject on a low-fat diet, for example, a diet that is low in omega-6 dietary fatty acids. In particular embodiments, the methods comprise administering to a subject on a low-fat diet an effective amount of a composition comprising an omega-3 fatty acid, wherein the omega-3 fatty acid is in the free acid form. Without being bound by any particular theory, fatty acids in the free acid form are more readily absorbed into the body of a subject (i.e., are more bioavailable) than fatty acid esters. Accordingly, the subject will not be required to be on a high fat diet, which could be contraindicated for the particular condition to be treated, in order to achieve an effective amount of circulating omega-3 fatty acids in the body.

In certain embodiments, the methods described herein further comprise a step of monitoring the lc-PUFA levels in the body of the subject. In specific embodiments, the lc-PUFA levels in the blood of the subject are monitored. In some embodiments, the levels of omega-6 lc-PUFAs are monitored. In other embodiments, levels of AA are monitored. In still other embodiments, the AA:DGLA ratio is monitored. In still other embodiments, the levels of omega-3 lc-PUFAs are monitored. In certain specific embodiments, EPA levels are monitored. In other specific embodiments, DHA levels are monitored. In yet other specific embodiments, the omega-3 index is monitored. In certain embodiments, the levels of omega-3 lc-PUFAs and the levels of omega-6 lc-PUFAs are monitored. In some embodiments, the AA:EPA ratio is monitored.

In particular embodiments, the methods described herein further comprise adjusting the dosage of omega-3 lc-PUFAs based on the lc-PUFA levels in the blood of the subject. In some embodiments, the dosage of omega-3 lc-PUFAs is adjusted based on the lc-PUFA levels in the red blood cells of the subject.

Levels of lc-PUFAs in the body of the subject can be ascertained by any method known in the art. Exemplary methods of monitoring lc-PUFA levels in a biological sample include, but are not limited to, chromatographic methods such as gas chromatography (GC), gas liquid chromatography (GLC), mass spectrometry (MS), high performance liquid chromatography (HPLC), reverse phase HPLC, thin layer chromatography (TLC), GC-MS and TLC-GLC, and the like, and spectroscopic methods such as nuclear magnetic resonance spectroscopy (NMR) and Fourier transform infrared spectroscopy (FTIR).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of identifying a subject who is susceptible to ADHD, comprising determining whether the subject is an efficient converter of mc-PUFA to lc-PUFA, wherein efficient converter status indicates susceptibility to ADHD.

2. The method of claim 1, wherein the determining step comprises measuring a ratio of arachidonic acid to dihomo-γ-linolenic acid in the body of the subject that is greater than about 6:1.

3. The method of claim 1, wherein the determining step comprises measuring an arachidonic acid level in the body of the subject that is greater than about 10% by weight of total fatty acids.

4. The method of claim 1, wherein the determining step comprises measuring a ratio of arachidonic acid to dihomo-γ-linolenic acid in the body of the subject that is greater than about 6:1 and measuring an arachidonic acid level in the body of the subject that is greater than about 10% by weight of total fatty acids.

5. The method of claim 1, wherein the determining step comprises measuring an omega-3 index in the red blood cells of the subject that is less than about 4% of total fatty acids.

6. The method of claim 1, wherein the determining step comprises measuring a ratio of arachidonic acid to eicosapentaenoic acid in the body of the subject that is greater than about 3.

7. The method of claim 1, wherein the determining step comprises detecting the presence of a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

8. The method of claim 1, wherein the determining step comprises:
a. measuring a ratio of arachidonic acid to dihomo-γ-linolenic acid in the body of the subject that is greater than about 6 or measuring an arachidonic acid level in the body of the subject that is greater than about 10% by weight of total fatty acids, and
b. detecting the presence of a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

9. The method of claim 1, wherein the subject has a clinical indication selected from the group consisting of oppositional defiant disorder, conduct disorder, antisocial personality disorder, borderline personality disorder, primary disorder of vigilance, a mood disorder, bipolar disorder, anxiety disorder, obsessive compulsive disorder, Tourette syndrome, a learning disorder and substance abuse.

10. A method of treating or preventing ADHD subject who is an efficient converter of mc-PUFAs to lc-PUFAs, comprising administering to a subject who has been determined to be an efficient converter of mc-PUFAs to lc-PUFAs an amount of a composition comprising omega-3 lc-PUFAs effective to treat or prevent ADHD.

11. The method of claim 10, wherein the subject has a ratio of arachidonic acid to dihomo-γ-linolenic acid in the body that is greater than about 6:1.

12. The method of claim 10, wherein the subject has an arachidonic acid level in the body that is greater than about 10% by weight of total fatty acids.

13. The method of claim 10, wherein the subject has a ratio of arachidonic acid to dihomo-γ-linolenic acid in the body that is greater than about 6:1 and has an arachidonic acid level in the body that is greater than about 10% by weight of total fatty acids.

14. The method of claim 10, wherein the subject has an omega-3 index in the red blood cells that is less than about 4% of total fatty acids.

15. The method of claim 10 wherein the subject has a ratio of arachidonic acid to eicosapentaenoic acid in the body that is greater than about 3.

16. The method of claim 10, wherein the subject has a single nucleotide polymorphism in a fatty acid desaturase gene selected from the group consisting of rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

17. The method of claim 10, wherein subject has:
a. a ratio of arachidonic acid to dihomo-γ-linolenic acid in the body that is greater than about 6:1 or an arachidonic acid level in the body that is greater than about 10% by weight of total fatty acids, and
b. has a single nucleotide polymorphism in a fatty acid desaturase gene selected from the group consisting of rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

18. The method of claim 10, wherein the subject has a clinical indication selected from the group consisting of oppositional defiant disorder, conduct disorder, antisocial personality disorder, borderline personality disorder, primary disorder of vigilance, a mood disorder, bipolar disorder, anxiety disorder, obsessive compulsive disorder, Tourette syndrome, a learning disorder and substance abuse.

19. The method of claim 10, wherein the composition comprises omega-3 lc-PUFAs in the ethyl ester form.

20. The method of claim 10, wherein the composition comprises omega-3 lc-PUFAs in the free acid form.

21. The method of claim 10, wherein the composition comprises omega-3 lc-PUFAs in the salt form.

22. The method of any one of claims 19-21, wherein the composition comprises EPA in an amount of at least about 45% by weight of fatty acids in the composition.

23. The method of claim 22, wherein the composition comprises EPA in an amount of at least about 55% by weight of fatty acids in the composition.

24. The method of claim 23, wherein the composition comprises EPA in an amount of at least about 75% by weight of fatty acids in the composition.

25. The method of claim 24, wherein the composition comprises EPA in an amount of at least about 90% by weight of fatty acids in the composition.

26. The method of any one of claims 19-21, wherein the composition comprises DHA in an amount of at least about 10% by weight of fatty acids in the composition.

27. The method of claim 26, wherein the composition comprises DHA in an amount of at least about 20% by weight of fatty acids in the composition.

28. The method of claim 27, wherein the composition comprises DHA in an amount of at least about 30% by weight of fatty acids in the composition.

29. The method of any one of claims 19-21, wherein the composition comprises EPA and DHA in a weight ratio of EPA to DHA of about 4.1:1.

30. The method of any one of claims 19-21, wherein the composition comprises EPA and DHA in a weight ratio of EPA to DHA of from about 1.24:1 to about 1.43:1.

31. The method of claim 29 or claim 30, wherein the EPA and DHA are in the ethyl ester form.

32. The method of any one of claims 19-21, wherein the composition comprises EPA in an amount of at least about 96% by weight of fatty acids in the composition, and no detectable DHA.

33. The method of claim 32, wherein the EPA is in the ethyl ester form.

34. The method of any one of claims 19-21, wherein the composition comprises no more than about 10% of omega-6 fatty acids by weight of fatty acids in the composition.

35. The method of claim 34, wherein the composition comprises no more than about 7% of omega-6 fatty acids by weight of fatty acids in the composition.

36. The method of claim 35, wherein the composition comprises no more than about 4% of omega-6 fatty acids by weight of fatty acids in the composition.

37. The method of claim 10, wherein the composition is administered orally.

38. The method of claim 10, wherein the composition is administered with a low fat meal.

39. The method of claim 10, wherein the amount of omega-3 lc-PUFAs effective to treat or prevent ADHD is at least 2 g/day.

40. The method of claim 39, wherein the amount of omega-3 lc-PUFAs effective to treat or prevent ADHD is at least 4 g/day.

41. A method of providing ADHD therapy to a subject in need thereof, comprising:
a. determining whether the subject is an efficient converter of mc-PUFA to lc-PUFA;
b. administering to the subject an effective amount of an ADHD therapy; and
c. adjunctively administering an amount of a composition comprising omega-3 lc-PUFAs effective to treat or prevent ADHD to the subject determined to be an efficient converter of mc-PUFA to lc-PUFA; and

42. In a method of providing ADHD therapy to a subject in need thereof, the improvement comprising:
a. determining whether the subject is an efficient converter of mc-PUFA to lc-PUFA;
b. administering to the subject an effective amount of an ADHD therapy; and
c. adjunctively administering with the ADHD therapy an amount of a composition comprising omega-3 lc-PUFAs effective to treat or prevent ADHD to the subject determined to be an efficient converter of mc-PUFA to lc-PUFA.

43. The method of claim 41 or claim 42, wherein the ADHD therapy is selected from the group consisting of amphetamine, methylphenidate, methamphetamine hydrochloride, dextroamphetamine, dexmethylphenidate, lisdexamfetamine dimesylate, atomoxetine, amantidine, modafinil, bupropion, venlafaxine, milnacipran, reboxetine, desipramine, nortriptyline, clonidine, guanfacine, and combinations thereof.

44. The method of claim 41 or claim 42, wherein the subject has a ratio of arachidonic acid to dihomo-γ-linolenic acid in the body that is greater than about 6:1.

45. The method of claim 41 or claim 42, wherein the subject has an arachidonic acid level in the body that is greater than about 10% by weight of total fatty acids.

46. The method of claim 41 or claim 42, wherein the subject has a ratio of arachidonic acid to dihomo-γ-linolenic acid in the body that is greater than about 6:1 and has an arachidonic acid level in the body that is greater than about 10% by weight of total fatty acids.

47. The method of claim 41 or claim 42, wherein the determining step comprises measuring an omega-3 index in the red blood cells of the subject that is less than about 4% of total fatty acids.

48. The method of claim 41 or claim 42, wherein the determining step comprises measuring a ratio of arachidonic acid to eicosapentaenoic acid in the body of the subject that is greater than about 3.

49. The method of claim 41 or claim 42, wherein the subject has a single nucleotide polymorphism in a fatty acid desaturase gene selected from the group consisting of rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

50. The method of claim 41 or claim 42, wherein subject has:
a. a ratio of arachidonic acid to dihomo-γ-linolenic acid in the body that is greater than about 6:1 or an arachidonic acid level in the body that is greater than about 10% by weight of total fatty acids, and
b. has a single nucleotide polymorphism in a fatty acid desaturase gene selected from rs174537, rs102275, rs174546, rs174556, rs1535, rs174576, rs174579, rs968567, rs173534, rs174549, rs174555, rs174556, rs174568, rs174567, rs498793, rs174545, rs174548 and combinations thereof.

51. The method of claim 41 or claim 42, wherein the subject has a clinical indication selected from the group consisting of oppositional defiant disorder, conduct disorder, antisocial personality disorder, borderline personality disorder, primary disorder of vigilance, a mood disorder, bipolar disorder, anxiety disorder, obsessive compulsive disorder, Tourette syndrome, a learning disorder and substance abuse.

52. The method of claim 41 or claim 42, wherein the composition comprises omega-3 lc-PUFAs in the ethyl ester form.

53. The method of claim 41 or claim 42, wherein the composition comprises omega-3 lc-PUFAs in the free acid form.

54. The method of claim 41 or claim 42, wherein the composition comprises omega-3 lc-PUFAs in the salt form.

55. The method of any one of claims 52-54, wherein the composition comprises EPA in an amount of at least about 45% by weight of fatty acids in the composition.

56. The method of claim 55, wherein the composition comprises EPA in an amount of at least about 55% by weight of fatty acids in the composition.

57. The method of claim 56, wherein the composition comprises EPA in an amount of at least about 75% by weight of fatty acids in the composition.

58. The method of claim 57, wherein the composition comprises EPA in an amount of at least about 90% by weight of fatty acids in the composition.

59. The method of any one of claims 52-54, wherein the composition comprises DHA in an amount of at least about 10% by weight of fatty acids in the composition.

60. The method of claim 59, wherein the composition comprises DHA in an amount of at least about 20% by weight of fatty acids in the composition.

61. The method of claim 60, wherein the composition comprises DHA in an amount of at least about 30% by weight of fatty acids in the composition.

62. The method of any one of claims 52-54, wherein the composition comprises EPA and DHA in a weight ratio of EPA to DHA of about 4.1:1.

63. The method of any one of claims 52-54, wherein the composition comprises EPA and DHA in a weight ratio of EPA to DHA of from about 1.24:1 to about 1.43:1.

64. The method of claim 62 or claim 63, wherein the EPA and DHA are in the ethyl ester form.

65. The method of any one of claims 52-54, wherein the composition comprises EPA in an amount of at least about 96% by weight of fatty acids in the composition, and no detectable DHA.

66. The method of claim 65, wherein the EPA is in the ethyl ester form.

67. The method of any one of claims 52-54, wherein the composition comprises no more than about 10% of omega-6 fatty acids by weight of fatty acids in the composition.

68. The method of claim 67, wherein the composition comprises no more than about 7% of omega-6 fatty acids by weight of fatty acids in the composition.

69. The method of claim 68, wherein the composition comprises no more than about 4% of omega-6 fatty acids by weight of fatty acids in the composition.

70. The method of claim 41 or claim 42, wherein the composition is administered orally.

71. The method of claim 41 or claim 42, wherein the composition is administered with a low fat meal.

72. The method of claim 41 or claim 42, wherein the amount of omega-3 lc-PUFAs effective to treat or prevent ADHD is at least 2 g/day.

73. The method of claim 72, wherein the amount of omega-3 lc-PUFAs effective to treat or prevent ADHD is at least 4 g/day.

74. The method of claim 41 or claim 42, wherein the composition is administered in advance of the ADHD therapy, and then concurrently with the ADHD therapy.

75. The method of claim 41 or claim 42, further comprising the step of monitoring the lc-PUFA levels in the blood of the subject.

76. The method of claim 75, wherein the lc-PUFA levels in the red blood cells of the subject are monitored.

77. The method of claim 75 or claim 76, wherein the lc-PUFA levels are monitored using gas chromatography.

78. The method of claim 75, wherein the levels of omega-6 lc-PUFAs are monitored.

79. The method of claim 78, wherein the levels of arachidonic acid are monitored.

80. The method of claim 78, wherein the ratio of arachidonic acid to dihomo-γ-linolenic acid is monitored.

81. The method of claim 75, wherein the levels of omega-3 fatty acids are monitored.

82. The method of claim 81, wherein the level of eicosapentaenoic acid is monitored.

83. The method of claim 81, wherein the level of docosahexaenoic acid is monitored.

84. The method of claim 75, wherein the omega-3 index is monitored.

85. The method of claim 75, wherein the omega-6 lc-PUFA levels and the omega-3 lc-PUFA levels in the blood of the subject are monitored.

86. The method of claim 85, wherein the ratio of arachidonic acid to eicosapentaenoic acid is monitored.

87. The method of claim 75, further comprising adjusting the dosage of omega-3 lc-PUFAs based on the lc-PUFA levels in the blood of the subject.
